(19) United States
(12) Patent Application Publication
(10) Pub. No.: US 2005/0070883 A1
(43) Pub. Date: Mar. 31, 2005

(54) Devices and methods for controlled delivery from a drug delivery device

(76) Inventors: James E Brown, Los Gatos, CA (US); John Culwell, Mountain View, CA (US); James A Filice, Los Gatos, CA (US); Edward M Gillis, Cupertino, CA (US); Felix Theeuwes, Los Altos Hills, CA (US); Peter Wickman, San Francisco, CA (US); Su Il Yum, Los Altos, CA (US)

Correspondence Address: DURECT CORPORATION, 10240 BUBB ROAD, CUPERTINO, CA, 95014, US

(21) Appl. No.: 10/433,196

(22) PCT Filed: Nov. 21, 2001
(86) PCT No.: PCT/US01/43143

Related U.S. Application Data
(60) Provisional application No. 60/250,328, filed on Nov. 29, 2000.

(51) Int. Cl. A61K 9/22

(52) U.S. Cl. 604/890.1

(57) ABSTRACT

The invention features a plug for use with a drug delivery device, wherein the plug defines an expansion control channel, which accommodates thermal expansion of a formulation in a reservoir of a drug delivery device, and an exit channel. In one embodiment, the plug comprises an inner plug member and an outer plug member, which members define an expansion control channel to facilitate release of entrapped air and to accommodate thermal expansion of formulation from the sealed drug reservoir. The plug further defines an exit channel, and may optionally further comprise a frit positioned within the flow pathway just prior to the delivery outlet, or both.

### FIELD OF THE INVENTION

The invention relates to devices and methods to facilitate controlled drug delivery.

### BACKGROUND OF THE INVENTION

Implantable drug delivery devices provide an attractive therapeutic tool for treatment of a variety of conditions and diseases, especially where therapy requires a prolonged period of therapy. Implantable drug delivery devices avoid the inconvenience and discomfort that can be associated with administration of multiple doses of an agent, and further provides for enhanced therapeutic benefits due to, for example, avoidance of bolus doses (e.g., in contrast to parenteral injection) and improved patient compliance. Devices that provide for precisely controlled drug delivery are of particular interest, as such devices can provide for delivery of drug at doses and rates that are both predictable and reliable (e.g., not affected by the environment in which the device is implanted).

Various implantable drug delivery devices have been developed, and are based upon various different mechanisms to accomplish movement of drug from a reservoir of the device to a treatment site in the subject. In general, these delivery technologies can be based upon, for example, diffusive, erodible, or convective mechanisms. Implantable drug delivery devices based upon convective systems are of particular interest in the field, generally due to advantageous features such as the ability to refill the device, and the compatibility of the device for use with a catheter to effect local delivery of drug to a treatment site. Exemplary convective systems include, but are not limited to, electromechanical pumps, osmotic pumps, electroosmotic pumps, electrochemical pumps, hydrolytic systems, piezoelectric pumps, elastomeric pumps, vapor pressure pumps, and electrolytic pumps.

The development of implantable devices, particularly for controlled delivery of drug, has posed several challenges in the drug delivery field. One such challenge is the ability to provide for precisely controlled delivery of drug even from the moment of start-up, e.g. the period just after implanting the drug delivery device. For example, variations in environmental temperature during storage and following implantation can cause expansion and contraction of the formulation, which in turn can affect the amount of formulation delivered following implantation. Expansion of the formulation following implantation can result in “extra dosing,” an uncontrolled release of a small amount of formulation, which can be particularly problematic where highly concentrated drug formulations are used. Environmental temperature shifts can also cause expansion and contraction of any air that may be trapped in the reservoir or between the reservoir and the outlet, which can also adversely affect the ability to provide for controlled delivery of drug formulation at start-up. In addition, the formulation flow can “split” during start-up, a phenomenon in which the formulation is not a substantially continuous stream of fluid, but rather is composed of one or more discrete leading volumes separated by air or gas voids (e.g., “burst(s)”) which precede the main formulation stream. As a result, at start-up the formulation may be delivered in a manner that is not precisely controlled.

One approach to solving these problems involves making the channel through which the formulation flows out of the reservoir small enough to regulate flow even when the formulation is in an expanded state. However, a channel small enough to regulate flow is generally unacceptably long (and, for example, unacceptably delaying delivery at start-up) or provides a volume insufficient to accommodate thermal expansion, which can result in leakage of formulation from the reservoir. Accurately filling a reservoir of a drug delivery device during manufacture in a manner that allows for capacity for the formulation to expand (e.g., due to variations in environment temperature during storage and following implantation) without loss of contents from the reservoir has proven extremely difficult.

As is evident from the above, there is a need for a device that can be used with drug delivery devices, particularly with convective drug delivery devices, that avoids the problems associated with drug delivery at start-up. The present invention addresses this problem.

### SUMMARY OF THE INVENTION

The invention features a plug for use with a drug delivery device, wherein the plug defines an expansion control channel, which accommodates thermal expansion of a formulation in a reservoir of a drug delivery device, and an exit channel. In one embodiment, the plug comprises an inner plug member and an outer plug member, which members define an expansion control channel to facilitate release of entrapped air and to accommodate thermal expansion of formulation from the sealed drug reservoir. The plug further defines an exit channel, and may optionally further comprise a frit positioned within the flow pathway just prior to the delivery outlet, or both.

In one aspect the invention features a plug comprised of an inner plug member and outer plug member adapted to receive the inner plug member, wherein the plug defines an inlet, an expansion control channel, and an outlet. The inlet, expansion control channel and outlet of the plug define a flow path through and out of the plug. In one embodiment, at least a portion of the expansion control channel defines a passageway through the inner plug member body. In another embodiment, the inner plug member is slidable within the expansion control channel. In related embodiments, the expansion control channel is defined by the outer plug member so that the slidable inner plug member is received within the outer plug member. In a related alternative embodiment, the expansion control channel is defined by adjacent ends of the inner plug member and the outer plug member and an inner wall of the reservoir body of the drug delivery device.

In another aspect, the invention features a drug delivery device comprising a plug of the invention. In one embodiment, the drug delivery device is implantable.

In still another aspect the invention features methods for delivery of a drug using a drug delivery device comprising a plug of the invention. In one embodiment, the drug delivery device is implantable.

A primary object of the invention is to provide for controlled delivery of drug while avoiding problems associated with conventional devices such as formulation leakage (e.g., due to thermal expansion of formulation during storage), delivery of a burst or bolus of formulation at start-up, and the like.

One advantage of the invention is that that the invention allows for accurate filling of a reservoir while maintaining an outflow track which serves to dampen the effects of thermal expansion of the fluid in the reservoir.

One important advantage of the invention is that the plug helps to prevent “extra dosing” or the initial, uncontrolled release of drug (e.g., “burst”) that can result from thermal expansion due to short-term changes in environmental temperatures.

Another advantage is that the invention minimizes or avoids entrapment of air during assembly, thereby minimizing or avoiding the problems such entrapped air can pose (e.g., due to the differences in the expansion rate of air compared to the expansion rate of formulation in the reservoir).

Another advantage of the invention is that it provides for precise control of start-up time for the drug delivery device.

Another advantage of the invention is that it provides for an extended outflow track while maintaining a more streamlined and volume efficient reservoir and delivery system size.

Still another advantage of the invention is that the plug allows for a smaller size reservoir to be filled and to withstand temperature variations, while still allowing for visual or other inspection to ensure a proper fill before completion of reservoir closure.

Another advantage of the invention is that it contains the drug formulation within the delivery device until the desired time for delivery, e.g., the plug inhibits leakage of formulation out of the reservoir during storage, shipping, etc.

These and other objects, advantages and features of the present invention will become apparent to those persons skilled in the art upon reading the details of the methodology and compositions as more fully set forth below.

### BRIEF DESCRIPTION OF THE DRAWINGS

FIG. 1 is a schematic of a convective delivery system.

FIG. 2 is a cut-away view of a plug of the invention inserted for use in a reservoir of a drug delivery device.

FIG. 3 is a cut-away view of a plug of the invention assembled for use with a drug delivery device.

FIG. 4 is a cut-away view of a plug of the invention comprising a frit positioned within the formulation flow path and prior to the outflow channel.

FIG. 5 is a cut-away view of a plug of the invention comprising a frit positioned with the flow path of the exit channel.

FIGS. 6-10 are cut-away views of the plug of the invention and a reservoir of a drug delivery device during insertion of the plug into the reservoir for use.

FIGS. 11 and 12 are cut-away views of an exemplary plug of the invention in which the inner plug member is slidable within an expansion control channel defined by the outer plug member.

FIGS. 13 and 14 are cut-away views of an exemplary plug of the invention in which the inner plug member is slidable within an expansion control channel defined by the outer plug member.

FIGS. 15 and 16 are cut-away views of an exemplary plug of the invention in which the inner plug member is slidable within an expansion control channel defined by the reservoir body.

FIGS. 17 and 18 are cut-away views of an exemplary plug of the invention in which the inner plug member is slidable within an expansion control channel defined by the reservoir body, where the inner plug member includes and O-ring.

### DESCRIPTION OF THE PREFERRED EMBODIMENTS

Before the present invention is described, it is to be understood that this invention is not limited to the specific devices, materials, formulations, or exemplary embodiments described as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “and”, and “the” include plural referents unless the context clearly dictates otherwise. Thus, for example, reference to “a drug delivery device” includes a plurality of such devices and reference to “the assembly method” includes reference to equivalent steps and methods known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

Definitions

The term “drug” as used herein is meant to encompass any substance suitable for delivery to a treatment site of a subject, which substances can include pharmaceutically active drugs, as well as biocompatible substances that do not exhibit a pharmaceutical activity in and of themselves, but that provide for a desired effect at a treatment site, e.g. to flush or irrigate a treatment site (e.g., saline).

The term “therapeutically effective amount” is meant an amount of a therapeutic agent, or a rate of delivery of a therapeutic agent, effective to facilitate a desired therapeutic effect. The precise desired therapeutic effect will vary according to the condition to be treated, the formulation to be administered, and a variety of other factors that are appreciated by those of ordinary skill in the art.

“Delivery site” as used herein is generally meant to refer to an area of the body to which drug is delivered for either local therapy or entry into the systemic circulation. Exemplary delivery sites include, but are not necessarily limited to, subcutaneous, intravenous, intra-arterial, intramuscular, intra-adipose tissue, and intra-lymphatic sites.

The term “implantation site” is used to refer to a site within the body of a subject at which a drug delivery device is introduced and positioned.

“Drug delivery device” as used herein is meant to any device, generally an implantable device, suitable for delivering a drug of choice, and in general suitable for use with the present invention. “Drug delivery device” thus encompasses any implantable device with any mechanism of action compatible with the claimed invention including, but not necessarily limited to, these convective systems (e.g., osmotic pumps, electromechanical pumps, electroosmotic pumps, electrochemical pumps, hydrolytic systems, piezoelectric pumps, elastomeric systems, vapor pressure pumps, and electrolytic pumps.

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not the environment of use.

The term “subject” is meant any subject, generally a mammal (e.g., human, canine, feline, equine, bovine, etc.), to which delivery is desired.

Overview of the Invention

The invention features devices and methods to provide for delivery of an agent from a drug delivery device while avoiding the problems associated with, for example, expansion and contraction of the formulation within the reservoir of the drug delivery device without allowing significant amounts or substantially any air into the sealed drug reservoir. Another requirement of this system was to provide an exit pathway from the device so as to allow regulation of flow of formulation through the channel, particularly at very low flow rates and in very low volumes. In general, the invention must provide a pathway small enough to accommodate regulated delivery of the formulation from a drug delivery device reservoir, but that at the same time is not unacceptably long or of insufficient volume to accommodate thermal expansion.

The present invention accomplishes these goals by providing a plug comprising inner and outer plug members, and defining a first channel of sufficient volume for thermal expansion (referred to herein as an expansion control channel) and a second, smaller channel (referred to herein as the exit channel) that provides for regulated delivery of the formulation from the reservoir without wicking of the formulation out of the reservoir or allowing seepage of fluids surrounding the drug delivery device into the plug and into the drug reservoir.

The invention will now be described in more detail.

Expansion Control Channel

In all embodiments, at least a portion of the plug defines an expansion control channel. The expansion control channel is of a volume sufficient to accommodate thermal expansion of a formulation to be delivered from a reservoir of a drug delivery device, and during delivery of formulation communicates with an exit channel to allow for flow of formulation from the reservoir and out an outlet of the device.

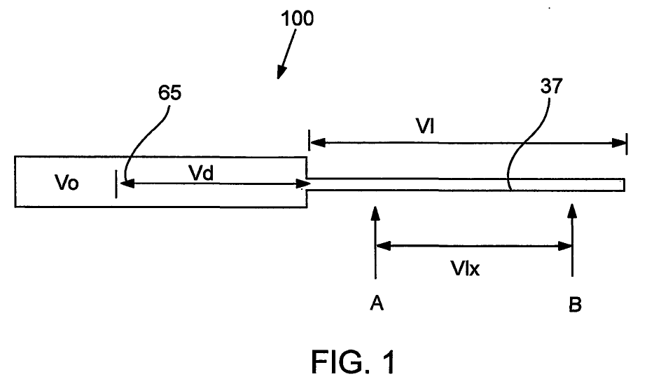

In considering the dimensions of the expansion control channel, one can assume a convective delivery system 100 (see schematic in FIG. 1) having a drug reservoir 65 and an orifice 37 (depicted in FIG. 1 as a linear path extending from reservoir 65). Reservoir 65 has a volume V_{d} with volume Vₒ as additional volume that may impact thermal expansion, Vₜ then the total volume that participates in thermal expansion can be expressed:
Vₜ=V_{d}+Vₒ (1)
Assume that delivery system 100 is filled with formulation to the mark A to extend into the linear orifice volume Vₗ. Mark A is reached at storage temperatures T_{S} and on implantation the volume expansion of the liquid reaches to the mark B at body temperature T_{B}. The rise of the volume expansion chamber Vₗₓ can then be calculated as follows. The volume expansion coefficient of the liquid formulation in the system is “a” as per the equation:
1/Vₜ(ΔV/ΔT)=a (2)
Assuming that a is constant over the temperature range, it then follows that the rise of the volume expansion chamber Vₗₓ can be calculated by the equation:
Vₗₓ=aVₜ(T_{B}−T_{S}) (3)
Thus a volume expansion chamber can be designed according to the invention by taking into account, for example, the values a (the volume expansion coefficient of the formulation to be delivered), Vₗ (the volume of the orifice, which varies with the length of the pathway through such orifice), (T_{B}−T_{S}) (the difference between the body (implantation) temperature T_{B} and the storage temperature T_{S}), the orifice diameter, and the length (B−A) along the orifice.

For example, for an organic drug formulation, aₒ=1.1×10⁻³/° C., and for an aqueous formulation aₐ=3×10⁻⁴/° C. According to equation (3), Vₗₓ for organic and water-based formulations, with a drug reservoir of V=0.200 cm³ and ΔT (or (T_{B}−T_{S}))=17° C., are:
Vₗₓ=a(0.2)(17)=a(3.4)
Vₗₓ=aₒ(3.4)=(1.1×10⁻³)(3.4)=3.7 μl for an organic liquid formulation, and
Vₗₓ=aₐ(3.4)=(3.1×10⁻⁴)(3.4)=1.0 μl for an aqueous formulation, and
   The system can also be designed to take into account the coefficient of linear expansion of the material through which the formulation flows. Values for the thermal expansion of formulations and components of such formulations are readily available and/or can be readily determined by methods well known in the art.

The expansion control channel can be provided by the plug of the invention in a variety of ways. For example, the expansion control channel can extend from one end of the inner plug member (a first end that will be in contact with the formulation of the drug reservoir during use), and through nearly all or a portion of the length of the inner plug member body to communicate with the exit channel of the plug. In one exemplary embodiment, the expansion control channel comprises a longitudinal channel which passes through the inner plug member body, a lateral passage, and a helical section. The longitudinal channel, which defines an inlet at a first end of the inner plug member (e.g., the end that is in contact with formulation in a reservoir during use), extends through the body of the inner plug member until it extends a desired distance toward the inner plug member second end. This longitudinal section can be positioned in the center of the inner plug member body, or may be off-center, as desired. The lateral section of the expansion control channel extends from the longitudinal section and to the helical section of the expansion control channel to provide a fluid passage between the longitudinal section to the outer wall. The helical section of the expansion control channel provides for fluid communication from the lateral section to the exit channel defined at least in part by the outer plug member (described below) or, where used in combination with a frit, to an outlet (described below). The helical section of the expansion control channel is defined at least in part by the inner plug member body. In one embodiment, the helical section of the expansion control channel is defined by the mating surfaces of the inner plug member outer wall and the outer plug member inner wall (e.g., by a groove in the inner plug member outer wall and an inner wall of the outer plug member). Each of these sections of the expansion control channel can be of varying dimensions, as will be readily appreciated by the ordinarily skilled artisan upon reading the instant disclosure.

In another embodiment, the expansion control channel is defined by the outer plug member body and an end of the inner plug member which is to be proximal to the reservoir during use. In this embodiment, the inner plug member is slidably received within the outer plug member body. Expansion of the formulation against the inner plug member proximal end causes the inner plug member to slide within the outer plug member, thereby providing an expansion control channel within the proximal end of the plug. The inner plug is prevented from moving out the distal end of the drug delivery device by the outer plug member, which is stably seated in the device.

In another embodiment, the inner plug member is slidable along the walls of the reservoir body of the drug delivery device, with the outer plug member being stably seated within the drug delivery device distal end. Expansion of the formulation against the inner plug member causes the inner plug member to slide within the reservoir body. outer plug member, thereby providing an expansion control channel within the proximal end of the plug. The outer plug member, which is again stably seated within the delivery device reservoir, prevents the inner plug from moving out the distal end of the drug delivery device.

The expansion control channel volume can be increased by varying the geometry of the channel. For example, the expansion control channel can be made longer by making all or a portion of the channel a spiral winding through or along the side of the inner plug member.

Inner Plug Member

The inner plug member of the plug of the invention is received by and at least a portion seated within the outer plug member. The inner plug member, generally either alone or in combination with the outer plug member, defines an expansion control channel, the conduit through which formulation will initially flow during use with a drug delivery device. The expansion control channel is of a size (diameter and length) that provides for control of air entrapment during assembly (e.g., insertion of the plug into a drug delivery device to provide for a sealed drug reservoir). In addition, the expansion control channel is of a size that accommodates thermal expansion of air and/or formulation so as to prevent or substantially diminish release of drug from the reservoir prior to start-up.

The size of the inner member can be minimized by defining the expansion control channel as a groove along an outside wall of the inner plug member or along an inside wall of the outer plug member, so that the complete expansion control channel is defined by the mating surfaces of the inner member outer wall and an inner wall of the outer plug member. In one embodiment, a first portion of the expansion control channel extends through the length of the inner plug member body, extends laterally through the inner plug member body, and then is defined as a helical groove that spirals along and down the outside wall of the inner plug member body such that the helical groove defines a complete expansion control channel when the inner member is positioned within the outer plug member. Suitable materials are hard plastics, metals, metal alloys, ceramics, polymers, and the like, where the materials provide for well-defined dimensions of the exit channel that can be maintained. It is desirable to have materials of low surface energy such that they prevent wicking of liquids through the channels. Materials such as metals can also be surface coated to reduce surface energy.

Outer Plug Member

In general, the outer plug member is the plug portion that defines the distal end of the plug, and further defines the outlet end of the drug delivery device when in use, e.g., the outer plug member provides at least a portion of the distal end of the drug delivery device, the end through which formulation is delivered to a delivery site or into a catheter operatively attached to the drug delivery device (e.g., by a press-fit, threaded, snap-fit, or adhesive attachment element and the like, and/or by welding, bonding, molding, and the like).

In one embodiment, the outer plug member is adapted to receive the inner plug member, which inner plug member may be either stably seated or slidable within the outer plug member. In another embodiment, the inner plug member is slidable within the reservoir body, and is prevented from being pushed out the drug delivery device by the outer plug member, i.e., the inner plug member slides through the reservoir until it contacts a proximal end of the outer plug member.

In one embodiment, the outer plug member defines, either alone or in combination with an inner wall of the drug delivery device, an exit channel through which the formulation flows out of the device during use. The exit channel is of a size that provides for regulated flow of formulation from the reservoir and the inner plug member channel during drug delivery, prevents wicking of formulation from the reservoir and out of the device, and prevents backflow of formulation or environmental fluids into the plug and reservoir during use. Backflow can have undesirable effects including contamination of the interior of the delivery device, and can result in dilution, destabilization, or other undesirable effect upon the formulation in the reservoir.

Parameters for the dimensions of an exit flow pathway that provides for controlled flow of a formulation through the pathway and with regulation of back-diffusion are described in, for example, U.S. Pat. Nos. 5,985,305 and 5,728,396. Briefly, the length, interior cross-sectional shape and area of the exit channel 41 are selected so that the average linear velocity of the released formulation is higher than that of the linear inward flux of materials in the environment of use due to diffusion or osmosis, so as to attenuate or moderate back-diffusion. The release rate of formulation (and thus of drug) can be modified by modifying the outlet pathway geometry, which are related as discussed below.

The convective flow of active agent out of outlet 45 is set by the delivery rate of the system and the concentration of drug A in the formulation 75 of reservoir 65. The relationship of these variables can be represented by the following formula:
Q_{ca}=(Q)(Cₐ) (4)
where Q_{ca} is the convective transport of drug A in mg/day; Q is the overall convective transport of the agent and its diluents in cm³/day; and Cₐ is the concentration of a drug in the formulation within reservoir Q_{ca} 65 in mg/cm³. The diffusive flow of drug through the material in the exit channel 41 is a function of drug concentration, cross-sectional configuration of the exit channel 41, drug diffusivity, and length of exit channel 41, and can be represented as follows:
Q_{da}=Dπr²ΔCₐ/L (5)
where Q_{da} is the diffusive transport of drug A in mg/day; D is the diffusivity through the material in exit channel 41 in cm²/day, r is the effective inner radius of the flow path in cm, ΔCₐ is the difference between the concentration of drug A in the reservoir and in the body outside of the outlet 45 in mg/cm³; and L is the length of the flow path in cm.

In general, the concentration of drug in the reservoir is much greater than the concentration of drug in the body outside of the outlet such that the difference, ΔCₐ, can be approximated by the concentration of agent within the reservoir, Cₐ.
Q_{da}=Dπr²Cₐ/L (6)
In general, the diffusive flux of drug is kept at less than 10% of the convective flow of drug. This is represented as follows:
Q_{da}/Q_{ca}=Dπr²Cₐ/QCₐL=Dπr²/QL≦0.1 (7)

For a delivery system with a purging rate of 1.5 μl/day, with a reservoir volume of 150 μl, where the system is to deliver the formulation over 100 days and has an orifice length of 1.1 cm and orifice diameter of 10 mil, assuming a diffusion coefficient of D=2×10⁻⁶ cm²/sec, the ratio of (Q_{da}/Q_{ca})=5.2×10⁻³ and satisfies equation (7). For this orifice size the volume Vₗ=5 μl and satisfies the criteria Vₗₓ<V₁ projected in FIG. 1. Exemplary ranges for Vₗₓ are then from 0.1 to 0.9 times Vₗ.

The exit channel generally extends through a side wall of the outer plug member body and is in fluid communication with the expansion control channel. The exit channel can extend nearly all or a portion of the length of the outer plug member body before providing an outflow channel for release of formulation out of the plug and device. An exit channel of desired length can be provided without requiring a concomitant increase in the dimensions of the outer plug member by varying the shape of the exit channel.

For example, the exit channel can be provided as a spiral extending along all or a portion of the outer plug member body. In one embodiment, the exit channel is provided as a groove along the outer wall of the outer plug member, with the complete exit channel provided by the mating surfaces of an inner wall of the drug delivery device (e.g., an inner wall of the drug reservoir) and an outer wall of the outer plug member following assembly of the plug for use with the device. In this embodiment, the diameter of the outer plug member may be minimized for use with smaller, implantable devices or microdevices. In another embodiment, the exit channel is provided by a combination of a channel through the outer plug member body that communicates with the expansion channel, and groove within the inner wall of the drug delivery device. In another embodiment in which the outer plug member is adapted to receive a slidable inner plug member, the exit channel can be defined by the mating surfaces of the outer plug member and the inner plug member, e.g., the exit channel can be defined by an outer wall of the inner plug member and an inner wall of the outer plug member.

Materials suitable for the outer plug member are the same as those suitable for use for the inner plug member. The materials used for the inner plug member and the outer plug member can be the same or different. Where the outer plug member is adapted to slidably receive the inner plug member, the inner wall of the outer plug member may be coated or other wise treated to facilitate sliding of the inner plug member.

Additional Plug Components or Elements

The plug of the invention can comprise other components that can serve to further enhance controlled delivery of drug through the plug. For example, in one embodiment, the plug comprises a frit positioned within the flow pathway so as to provide a void volume. The frit is generally positioned within the outer member and just prior to an outflow channel of the plug. In general, the frit comprises a porous material that is positioned within the path through which the formulation must pass, and which provides a void volume sufficient to trap liquid and allow air to escape. In use, the frit “captures” the leading edge of formulation flow (the “burst”) at start-up, and traps this formulation volume until the main body of formulation joins the initial formulation burst. Where the plug comprises a frit, the outer member plug may optionally define an exit channel as described above, e.g., where a frit is positioned with in the flow pathway, the exit may not be required to provide for the desired level of controlled delivery.

It will be readily appreciated that the materials suitable for use in the frit will vary according to a variety of factors including, but not necessarily limited to, the formulation to be passed through the frit (e.g., the formulation pH, hydrophobicity, hydrophilicity, wetting characteristics, and the like). In general, the frit material is selected so that it is capable of wetting and wicking the formulation, and is both compatible and chemically resistant to the formulation. Suitable frit materials include, but are not necessarily limited to, metals, glass, polymers, and cellulose.

Likewise, the dimensions of the frit and, thus the void volume provided by the frit, will vary according to a variety of factors that will be readily appreciated by the ordinarily skilled artisan. The frit materials are typically made with materials of high surface energy, such that they wet easily. In general, the frit materials are porous elements with porosities in the range of 30% to 90%, preferably 50% to 90%, with open pore structure. The internal volume of the porous element is in the range of 0.1 to 0.5 times Vₗₓ as defined in FIG. 1. In general, the volume provided by the frit is selected so that it can handle either the thermal expansion volume of the formulation (Vₗₓ), and may also be designed to handles any volume of fluid which may break away from the main stream of fluid exiting the pump, e.g., handling a small volume (e.g., 1-2 μl) of fluid which breaks from the fluid front and wick forward toward the plug outlet.

Drug Delivery Devices for Use with the Invention

Any of a variety of drug delivery devices are compatible for use with the invention. In general, drug delivery devices suitable for use with the plug of the invention are those comprising a drug reservoir that is designed to contain a flowable drug formulation, and which further comprises a portion that can receive the plug to provide for sealing of the drug reservoir by the plug, e.g., the plug can be positioned in the drug delivery device so as to provide for a flow pathway from the drug reservoir and out of the device.

The drug delivery device is generally capable of carrying a drug formulation in such quantities and concentration as therapeutically required, and of providing sufficient protection to the formulation from attack by body processes for the duration of implantation and delivery. The exterior is thus preferably made of a material that has properties to diminish the risk of leakage, cracking, breakage, or distortion so as to prevent expelling of its contents in an uncontrolled manner under stresses it would be subjected to during use, e.g., due to physical forces exerted upon the drug release device as a result of movement by the subject or physical forces associated with pressure generated within the reservoir associated with drug delivery. The drug reservoir or other means for holding or containing the drug must also be of such material as to avoid unintended reactions with the active agent formulation, and is preferably biocompatible (e.g., where the device is implanted, it is substantially non-reactive with respect to a subject's body or body fluids).

Suitable materials for the reservoir body or drug holding means of the drug delivery devices of the invention are well known in the art. For example, the reservoir material may comprise a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polyurethane, polychlorotrifluoroethylene, copolymer tetrafluoroethylene and hexafluoropropylene; polyethylene vinylacetate (EVA), polyimide; polysulfone; polycarbonate; polyethylene; polypropylene; polyvinylchloride-acrylic copolymer; polycarbonate-acrylonitrile-butadiene-styrene; polystyrene; cellulosic polymers; and the like. Further exemplary polymers are described in The Handbook of Common Polymers, Scott and Roff, CRC Press, Cleveland Rubber Co., Cleveland, Ohio. Metallic materials suitable for use in the reservoir body include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys. Laminates of the above materials can also be used in the reservoir body.

Where the drug formulation is stored in a reservoir comprising metal or a metal alloy, particularly titanium or a titanium alloy having greater than 60%, often greater than 85% titanium is preferred for the most size-critical applications, for high payload capability and for long duration applications and for those applications where the formulation is sensitive to body chemistry at the implantation site or where the body is sensitive to the formulation. Most preferably, the drug delivery devices are designed for storage with drug at room temperature or higher.

Drug delivery devices suitable for use with the invention can be based on any of a variety of modes of operation. In general, the plug of the invention can be used with any drug delivery device comprising a reservoir for containing a flowable drug formulation, and from which the formulation is to be delivered to a treatment site in the subject. The plug of the invention finds particular use with a convective device comprising a drug reservoir. Exemplary devices include, but are not necessarily limited to, electromechanical pumps, osmotic pumps, electroosmotic pumps, electrochemical pumps, hydrolytic systems, piezoelectric pumps, vapor pressure pumps, and electrolytic pumps. Drug release devices based upon a mechanical or electromechanical infusion pump, can also be suitable for use with the present invention. Examples of mechanical or electromechanical infusion pumps include, but are not necessarily limited to, those described in, for example, U.S. Pat. Nos. 4,692,147; 4,360,019; 4,487,603; 4,360,019; 4,725,852, and the like. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and 5,985,305; in PCT Publication No. WO 97/27840; and the like.

The plug of the invention is particularly useful where the drug delivery device is to provide for controlled delivery of drug, especially in very small amounts (e.g., 0.01 μg/hr to about 200 μg/hr) and/or low volumes (e.g., a volume rate of from about 0.01 μl/day to about 100 μl/day (i.e., from about 0.0004/to about 4 μl/hr), preferably from about 0.04 μl/day to about 10 μl/day, generally from about 0.2 μl/day to about 5 μl/day, typically from about 0.5 μl/day to about 1 μl/day). In one embodiment, the volume/time delivery rate is substantially constant (e.g., delivery is generally at a rate ± about 5% to 10% of the cited volume over the cited time period.

In some embodiments it may be desirable to provide a drug delivery catheter with the drug delivery device, e.g., where the implantation site and the desired delivery site are not the same or adjacent. The drug delivery catheter is generally a substantially hollow elongate member having a first end (or “proximal” end) associated with the drug delivery device, and a second end (or “distal” end) for delivery of the drug-comprising formulation to a desired delivery site. Where a drug delivery catheter is used, a first end of the drug delivery catheter is associated with or attached to the drug delivery device so that the lumen of the drug delivery catheter is in communication with, via the channels defined by the plug of the invention, the drug reservoir in the drug delivery device, so that a formulation contained in a drug reservoir can move into the drug delivery catheter, and out a delivery outlet of the catheter which is positioned at the desired delivery site.

The body of the catheter defines a lumen, which lumen is to have a diameter compatible with providing leak-proof delivery of drug formulation from the drug delivery device. Where the drug delivery device dispenses drug by convection (as in, e.g., osmotic drug delivery systems), the size of the catheter lumen leading from the reservoir of the drug release system can be designed as described by Theeuwes (1975) J. Pharm. Sci. 64:1987-91 expressed in equation (7). The body of the catheter can be of any of a variety of dimensions and geometries (e.g., curved, substantially straight, tapered, etc.), that can be selected according to their suitability for the intended site for drug delivery. The distal end of the drug delivery catheter can provide a distinct opening for delivery of drug, or as a series of openings.

Formulations for Delivery

Any of a variety of formulations comprising any of a variety of drugs (active agents) can be delivered using a drug delivery device comprising a plug of the invention. Classes of drugs suitable for delivery using an drug delivery device comprising a plug of the invention include, but are not necessarily limited to, pharmacologically active peptides, polypeptides, nucleic acid encoding a gene product of interest and such gene products (e.g., DNA, RNA, and other nucleic acid-based compounds), Compounds of interest include chemotherapeutic agents for neoplastic tissues, anti-inflammatory agents (e.g., for ischemic or inflamed tissues), hormones or hormone antagonists (e.g., for endocrine tissues), ion channel modifiers (e.g., for cardiovascular or other tissues), and neuroactive agents (e.g., for the central nervous system, including, but not necessarily limited to analgesics, including, but not limited to opioids, opioid derivatives, and the like). Exemplary of pharmaceutical agents suitable for this invention are those described in The Pharmacological Basis of Therapeutics, Goodman and Gilman, McGraw-Hill, New York, N.Y., (1993) under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

Exemplary Embodiments

Exemplary embodiments of the plug of the invention, as well as its use in connection with a drug delivery reservoir, will now be provided with reference to the drawings.

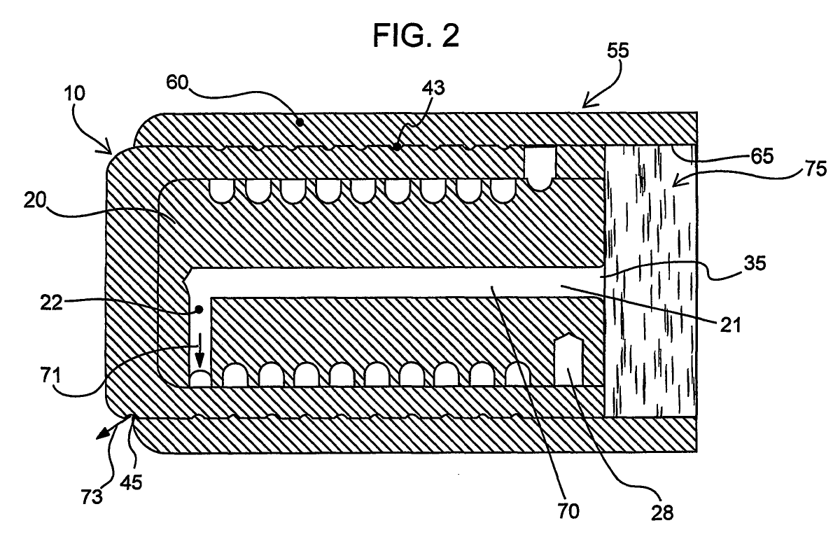

FIG. 2 shows a cut-away view of a plug 10 of the invention inserted for use within a reservoir 65 containing formulation 75, and defined by reservoir body 60 of drug delivery device 55. Plug 10 comprises inner plug member 20 and outer plug member 40. Inner plug member 20 is positioned within outer plug member 40. In this embodiment, expansion control channel 21 is composed of a longitudinally extending section, which extends from a first end of inner plug member 20 (which end defines inlet 35) and through the body of the inner plug member; a laterally extending section, which extends from the body of the inner plug member 20 and to an outer wall of the inner plug member 20, and a helical portion, which in this embodiment is defined by the mating surfaces of inner plug member 20 and outer plug member 40, specifically in this embodiment by the outer wall of inner plug member 20 (exemplified in FIG. 2 as a groove) and an inner wall of outer plug member 40.

Plug 10 of FIG. 2 further defines exit channel 41, which in this embodiment is defined by the mating surfaces of an outer wall of outer plug member 40 and an inner wall of reservoir body 60. Exit channel 41 communicates with expansion control channel 21 via passage or “gate” 30 through a wall of outer plug member 40. Timing hole 28, represented by a dead-end passage at the end of inner plug member 20 adjacent reservoir 65 during use and positioned opposite gate 30, facilitates manufacture and assembly of the plug to ensure that expansion control channel 21 and exit channel 41 are in fluid communication. In use, formulation 75 flows from reservoir 65 into inlet 35, through expansion channel 21, through exit channel 41, and out outlet 45, which in this embodiment is defined by an outer wall of outer plug member 40 and an inner wall of as illustrated by the flow pathway denoted by arrows 70, 71, 72, and 73.

The seal between the mating surfaces of the plug 10 and the reservoir body 60 can be designed to withstand the maximum pressure of the formulation generated within the device. In general, the pressure required to dislodge the plug 10 from the reservoir 65 is at least about ten times the pressure in reservoir 65.

In one embodiment, a substantially drug-free, immiscible, flowable material is provided within exit channel 41. The material is immiscible with the formulation to be delivered from reservoir 65. The immiscible material can be, for example, a solid at room temperature or other storage temperature, and extrudable from the exit channel at body temperature such that the immiscible material is extruded out the exit channel and out the outlet following implantation of the device. The immiscible material thus facilitates prevention of back diffusion into the plug and reservoir.

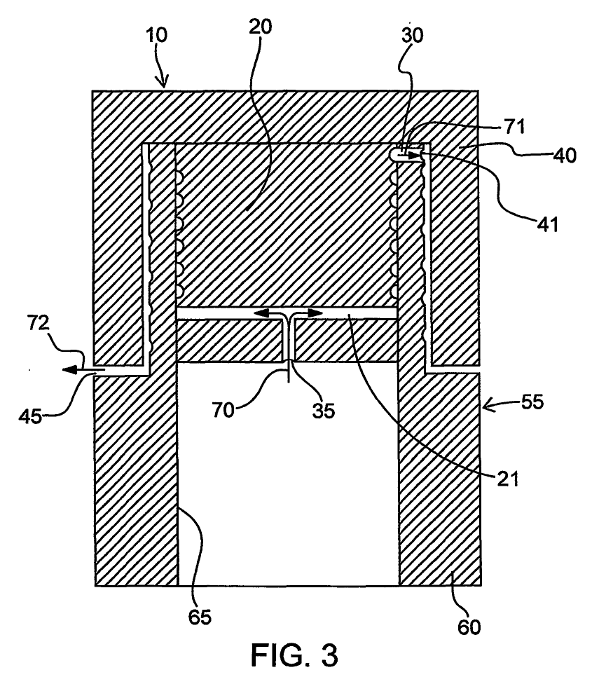

In another embodiment, exemplified in FIG. 3, exit channel 41 is defined by the mating surfaces of an outer wall of reservoir body 60 and an inner wall of outer plug member 40, where outer plug member 40 is adapted and sized to receive at least a portion of the reservoir body 60 and inner plug member 20 which is seated within reservoir body 60 (e.g., outer plug member 40 can fit over the end of reservoir body 60 in a cap configuration). Expansion channel 21 is defined by at least a portion of inner plug member 20; in the embodiment exemplified in FIG. 3, expansion channel 21 is defined by the mating surfaces of an outer wall of inner plug member 20 and an inner wall of reservoir body 60. As illustrated by arrows 70, 71, and 72, in this embodiment, the formulation moves through a flow pathway that passes through inlet 35, through expansion channel 21, through gate 30, through exit channel 41 and out outlet 45.

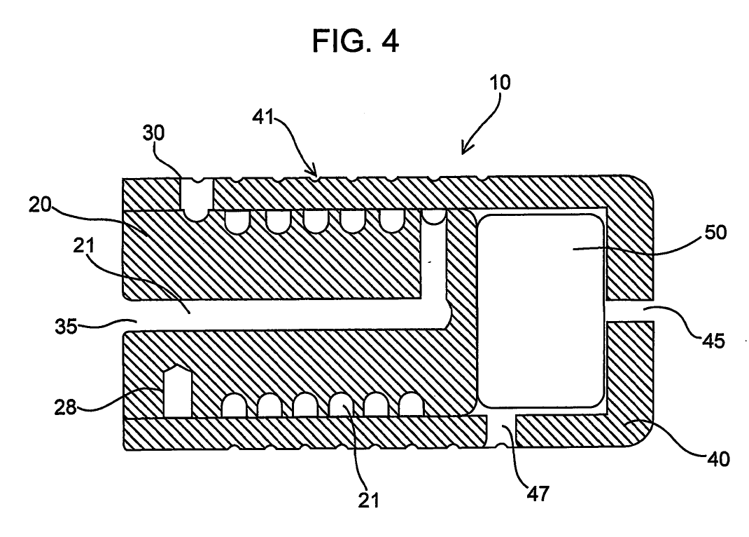

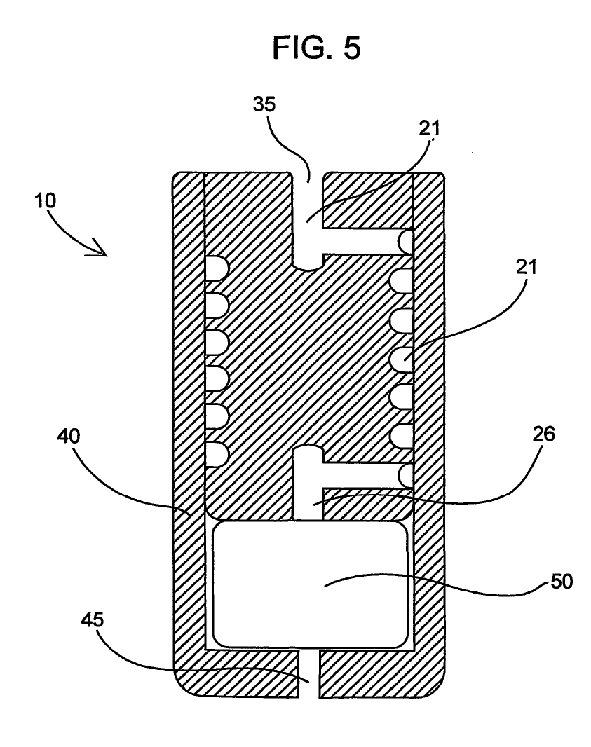

FIGS. 4 and 5 provide exemplary embodiments of the plug of the invention, where the plug comprises a frit positioned within the flow pathway. In the embodiment of FIG. 4, frit 50 is seated within outer plug member 40 just prior to exit of the flow pathway outer outlet 45. Outer plug member 40 comprises an additional passage 47 which provides for fluid communication between exit channel 41 (which in this embodiment is provided in part by a groove in an outer wall of outer plug member 40) and frit 50. When plug 10 is positioned for use in a reservoir of a drug delivery device, formulation flows into inlet 35, through expansion control channel 21, through a first outer plug member wall passage or “gate” 30, through exit channel, 41, through a second outer plug member wall passage 47, into frit 50, and out outlet 45.

In another embodiment, exemplified in FIG. 5, plug 10 comprises inner plug member 20, outer plug member 40, frit 50, and defines expansion control channel 21. The positioning of the frit in this embodiment avoids the need for an exit channel since the frit provides for regulated flow of formulation. Thus, formulation flows into inlet 35, through expansion control channel 21, through inner plug member passage 26, and into frit 50.

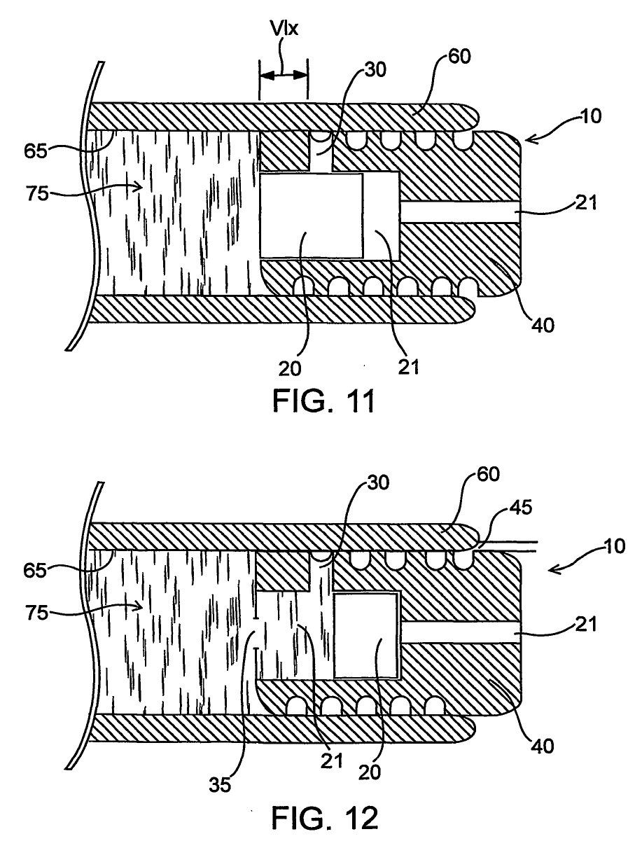

In another embodiment of the invention, illustrated in FIGS. 11-16, inner plug member 20 is slidably positioned within the expansion control channel. Inner plug member 20 is positioned after fill and prior to delivery so that gate 30 is closed, thereby preventing fluid communication between expansion control channel 21 and exit channel 41. Inner plug member 20 is slidable a distance within expansion control channel 21 so as to make available a volume of expansion control channel 21 sufficient to accommodate thermal expansion of formulation 75 (represented by Vlx). When delivery of formulation is desired, formulation pushes against the proximal end of inner plug member 20 so as to displace inner plug member 20 to create an inlet 35, and to displace inner plug member 20 a distance sufficient to slide the proximal end of inner plug member 20 past gate 30. Sliding of inner plug member 20 “opens” gate 30, providing for fluid communication between, and thus defining a flow pathway through, reservoir 65, expansion control channel 21, gate 30, exit channel 41, and outlet 45.

In a preferred embodiment, exemplified in FIGS. 11 and 12, the walls of expansion channel 21 are defined by the body of outer plug member 40. In this embodiment, the walls are substantially smooth, allowing for smooth sliding of inner plug member 20 along the walls of expansion channel 21. As in the embodiment described above, sliding of inner plug member 20 provides for an expansion control channel 21 of a volume sufficient to accommodate thermal expansion of formulation 75 (represented by Vlx). When delivery of formulation is desired, formulation pushes against the proximal end of inner plug member 20 so as to displace inner plug member 20 to create an inlet 35, and to displace inner plug member 20 a distance sufficient to slide the proximal end of inner plug member 20 past gate 30. Sliding of inner plug member 20 “opens” gate 30, providing for fluid communication between, and thus defining a flow pathway through, reservoir 65, expansion control channel 21, gate 30, exit channel 41, and outlet 45.

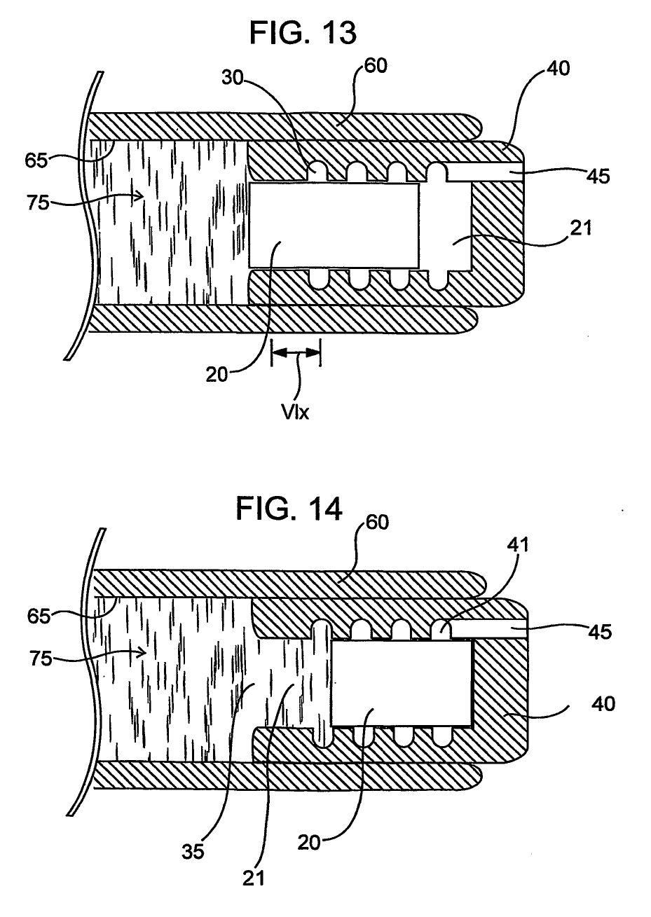

Exit channel 41 is generally defined by the mating surfaces of an outer wall of outer plug member 40 and an inner wall of reservoir body 60. The exit channel is exemplified in FIGS. 11 and 12 as being defined by a groove on an outer wall of outer plug member 30 and an inner wall of reservoir body 60. Alternatively, the exit channel can be defined by an outer wall of the outer plug member and grooves along the inner wall of the reservoir body. In a related embodiment, exemplified in FIGS. 13 and 14, exit channel 41 is defined by the mating surfaces of an outer wall of inner plug member 20 and an inner wall of expansion control channel 21. In this exemplary embodiment, outlet 45 is defined by the body of outer plug member 40.

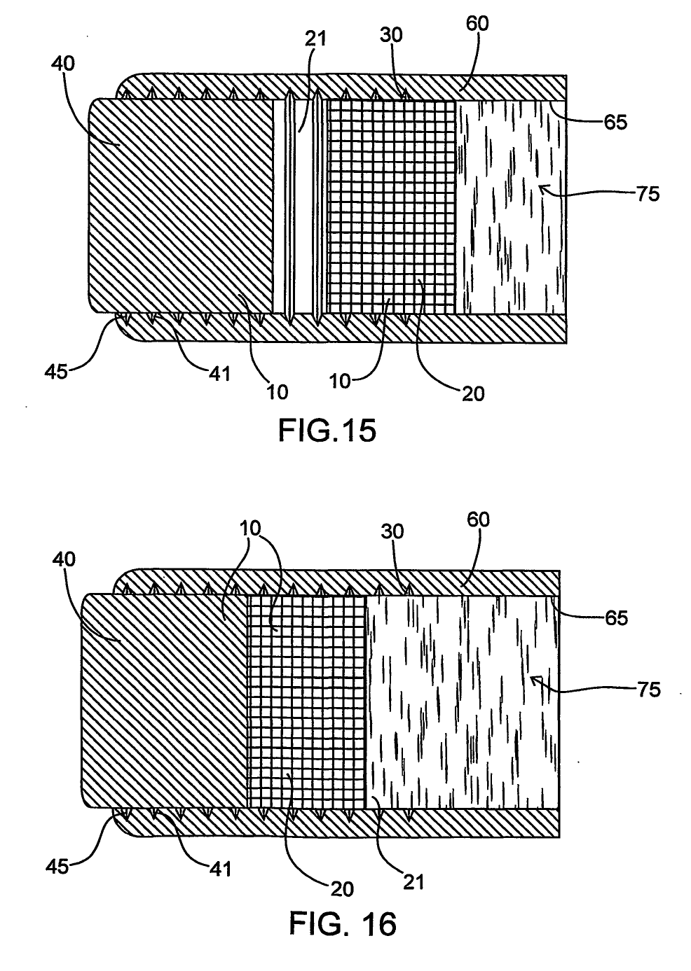

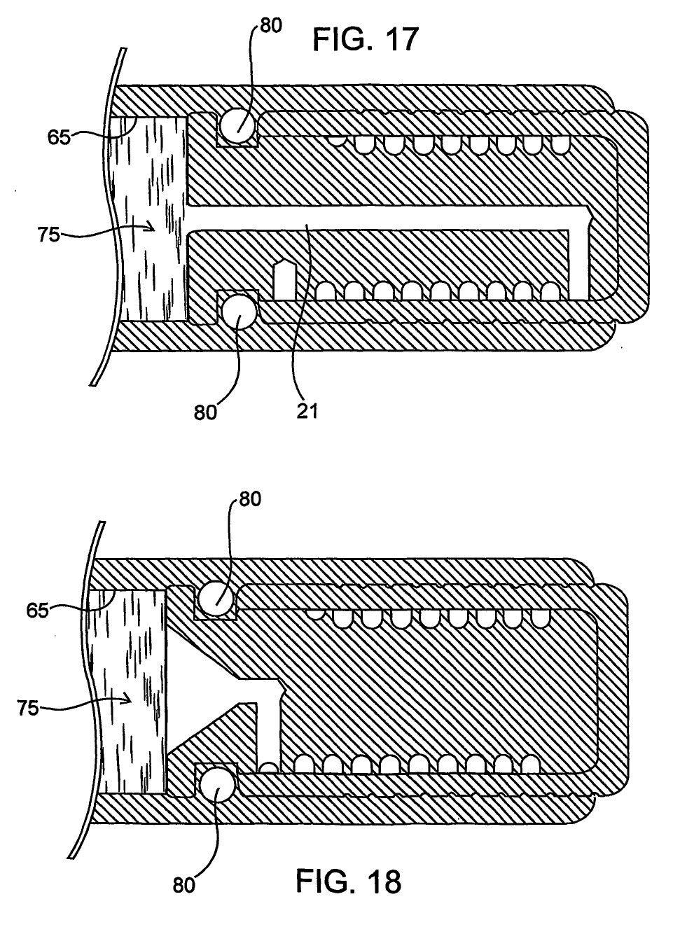

In another embodiment, exemplified in FIGS. 15 and 16, inner plug member 20 and outer plug member 40 are provided as separate components positioned within reservoir body 60. Outer plug member 40 is stably seated within reservoir body 65, while inner plug member is slidably positioned within expansion control channel 21 and at a position proximal to outer plug member 40 and adjacent formulation 75. As in the other embodiments described above, thermal expansion of formulation 75 forces movement of inner plug member 20 to provide a volume of expansion control channel 21 sufficient to accommodate thermal expansion (volume Vlx). At start-up of delivery, formulation 75 is forced against the proximal end of inner plug member 20 until gate 30 is opened to bring expansion control channel 21 into fluid communication with exit channel 41. In this example, exit channel 41 is defined by the mating surfaces of an outer wall of inner plug member 20, an outer wall of outer plug member 40, and an inner wall of reservoir body 60. In the embodiment shown in FIGS. 17 and 18, the inner plug member 20 further comprises an O-ring 80 to help prevent fluid leakage of formulation 75 from reservoir 65 into expansion channel 21.

Assembly and Use

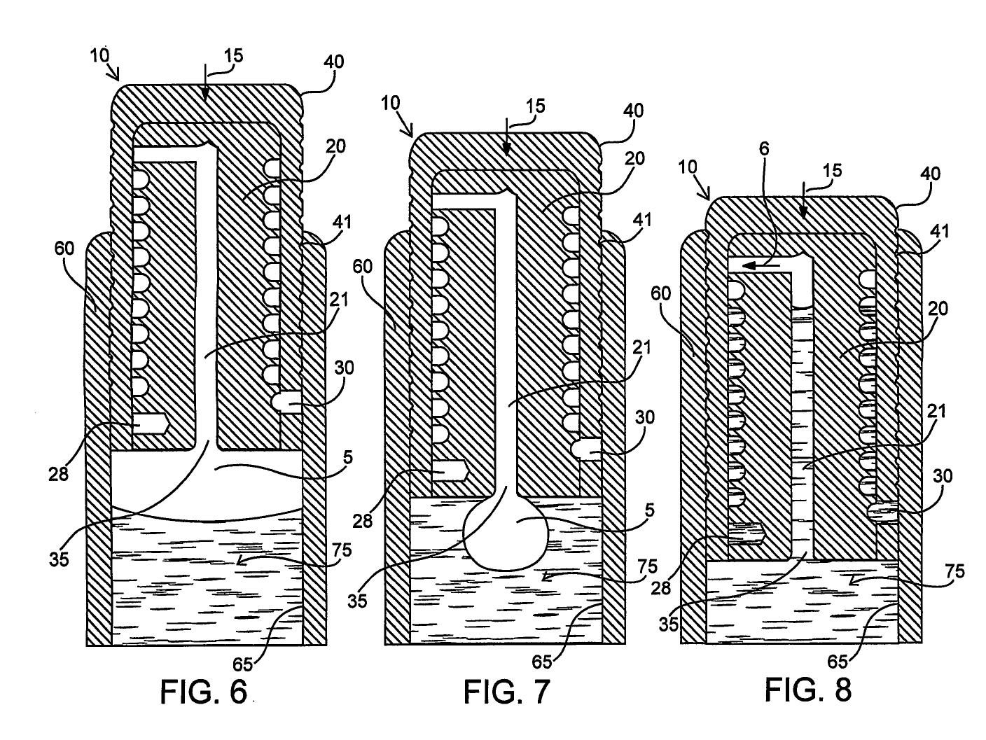

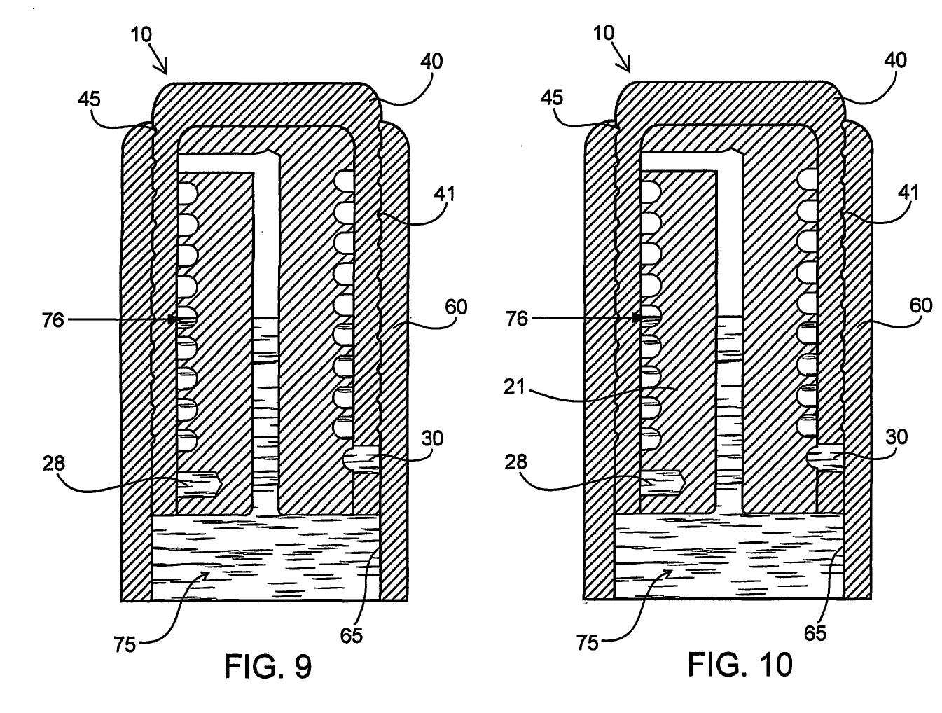

FIGS. 6-10 illustrate insertion of a plug of the invention for use with a drug delivery device. Reservoir 65 is filled with formulation 75 and plug 10 inserted into reservoir 65 as shown by moving plug 10 in the direction of arrow 15. Air 5 entrapped during insertion of plug 10 moves through inlet 35 inner plug member 20, through expansion control channel 21, through exit channel 41 and out of an opening from between an inner wall of reservoir 60 and an outer wall of outer plug member 40. Flow of air 5 out of plug 10 and out of the drug delivery device (indicated in FIG. 8 by arrow 6) is followed by flow of formulation into inlet 35 and into expansion channel 21. As the formulation fluid level (indicated by arrow 76) rises, e.g., due to thermal expansion of formulation during storage, formulation moves through expansion channel 21, and can move into exit channel 41, but without movement of any or any substantial amount through outlet 45, as illustrated in FIGS. 9 and 10.

Assembly of a drug delivery device such as that exemplified in FIG. 3 can be accomplished in a two-step plug assembly method. Assembly begins with reservoir 65 being filled with formulation 75 so that, for example, at room temperature (filling temperature of about 22° C.) the formulation will substantially completely fill the reservoir and plug to the top following insertion of inner plug member 20. For assembly, inner plug member 20 is first inserted into reservoir 65, preferably in a manner that allows for movement of any entrapped air through expansion control channel 21 and out of the system. Outer member plug 40 is then positioned over the assembled inner plug member 20 and reservoir body 60.

Assembly of plugs exemplified in FIGS. 11-14 can be accomplished in a one-step process by insertion of the plug into the distal end of a reservoir of a drug delivery device.

The plug may be inserted prior to or after filling of the reservoir with formulation. After assembly and filling of the reservoir, the inner plug member 20 is slidably positioned within expansion control channel 21 such that gate 30, which connects expansion control channel 21 with exit channel 41, is closed, e.g., is blocked by fluid communication with exit channel 41 by inner plug member 20. Thermal expansion or formulation 75 within the reservoir pushes against the proximal end of inner plug member 20. When the force of the formulation against inner plug member 20 is sufficient, inner plug member 20 slides through expansion control channel 21 to accommodate expanding formulation 75. Expansion control channel 21 is of a volume sufficient to accommodate thermal expansion without allowing for opening of gate 30 and thus without allowing for flow of formulation into exit channel 41. As illustrated in FIGS. 11 and 12, expansion control channel 21 can extend through a distal end of outer plug member 40 to allow for release of air that may be entrapped in the reservoir and to enable the inner plug member 20 to slide freely through the outer plug member 40 without a build up of pressure in expansion control channel 21. In the embodiments of FIGS. 13 and 14, the exit channel 45 can provide for release of air to relieve pressure during sliding of inner plug member 20.

At start-up of delivery of formulation from the device, formulation is forced against the proximal end of inner plug member 20 (see, e.g., FIGS. 12, 14, and 16). The force of formulation 75 against the inner plug member proximal end causes inner plug member 20 to slide through expansion control channel 21 until gate 30 is open. Opening of gate 30 allows formulation 75 to flow into exit channel 41 and out outlet 45.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1

A prototype plug as exemplified in FIG. 2 was manufactured. The inner plug member was machined from titantium, and was approximately 2.2987 mm in diameter and 5.062 mm in length. The longitudinal portion of the expansion control channel (inner diameter of 0.43 mm) was provided through the center of the inner plug member to a length of 4.689 mm. The lateral portion of the expansion control channel of 0.254 mm diameter was provided to the outside of the inner plug member body. The inner plug member outer wall defined grooves (0.400 mm pitch, 0.330 mm depth, 0.279 width) to further extend the expansion control channel.

The outer plug member was also machined from titanium. The outer plug member had an external diameter of 0.1185 mm, and defined a chamber for receiving the inner plug member. The receiving chamber had a depth of 0.200 mm and an inner diameter of 0.090 mm. The gate (i.e., the passage from the inner wall of the outer plug member to the outer wall of the outer plug member) was provided as a hole of 0.0145 mm in diameter. Grooves (0.0157 mm pitch, 0.0020 mm depth, and 0.0028 mm width) on the outer wall of the outer plug member were provided so as to define an exit channel when in use in a reservoir body.

The reservoir body into which the plug is to be inserted is approximately 3.150 mm in diameter, and approximately 43.18 mm in length. The components were press fit together.

The plug was tested by filling the reservoir of the device, and placing the device with the plug in place in a 37° C. water bath to simulate implantation in the body. Sample devices made with this plug showed no aberrant release of formulation upon placement into the water bath (i.e., release of formulation associated with thermal expansion of the formulation the device), indicating that the plug prevented the undesirable “burst” of drug that can be accompanied with implantation without the plug of the invention. In contrast, devices without the plug of the invention released about 1.5 μl of formulation immediately upon implantation, which release is not consistent with the desired delivery pattern.

1. A plug defining an inlet, an expansion control channel, and an outlet, wherein during use the inlet, expansion control channel and outlet define a flow path from a reservoir of a drug delivery device, through the plug, and out of the drug delivery device.

2. The plug of claim 1, wherein the plug comprises:
an inner plug member; and
an outer plug member.

3. The plug of claim 2, wherein the outer plug member is adapted to receive the inner plug member.

4. The plug of claim 2, wherein the inner plug member is slidable within the expansion control channel.

5. The plug of claim 2, wherein the expansion control channel is defined by the outer plug member.

6. The plug of claim 2, wherein the expansion control channel is defined by an inner wall of the drug delivery device.

7. The plug of claim 4, wherein during use the inner plug member is slidable to a first position to accommodate thermal expansion of a formulation in the drug delivery device reservoir.

8. The plug of claim 4, wherein the inner plug member is slidable to a second position to provide for fluid communication between the expansion control channel and the outlet.

9. The plug of claim 8, wherein the plug further defines an exit channel positioned between the expansion control channel and the outlet.

10. The plug of claim 2, wherein at least a portion of the expansion control channel is defined by an outer wall of the inner plug member and an inner wall of the outer plug member.

11. The plug of claim 2, wherein the plug further comprises a frit positioned in the flow path prior to the outlet.

12. The plug of claim 1, wherein the plug further defines an exit channel positioned in the flow path between the expansion control channel and the outlet.

13. The plug of claim 12, wherein the exit channel is at least partially filled with an drug formulation-immiscible fluid.

14. The plug of claim 12, wherein the exit channel is defined by a groove in an outer wall of the outer plug member and an inner wall of a reservoir following insertion of the plug into a reservoir of a drug delivery device.

15. The plug of claim 12, wherein the plug further comprises a frit positioned in the flow path between the exit channel and the outlet.

16. The plug of claim 2, wherein the expansion control channel comprises a first channel extending longitudinally through the inner plug member, a second channel extending laterally through the inner plug member, and a third helical channel extending in a spiral fashion along or through the inner plug member.

17. The plug of claim 16, wherein the third helical channel is defined by mating surfaces of an outer wall of the inner plug member and an inner wall of the outer plug member.

18. A plug comprising,
an inner plug member comprising a first end and a second end and an inner plug member body; and
an outer plug member adapted to receive the inner plug member;
wherein the plug defines an expansion control channel, the expansion control channel extending from a plug inlet, through the inner plug member body, and to an outlet defined by the outer plug member, and wherein the inlet, expansion control channel, and outlet define a flow path through the plug.

19. The plug of claim 18, wherein the expansion control channel is defined by the outer plug member, and the inner plug member is slidable within the expansion control channel.

20. The plug of claim 19, wherein the inner plug member is slidable to a first position to accommodate thermal expansion of a formulation in the drug delivery device reservoir.

21. The plug of claim 19, wherein the inner plug member is slidable to a second position to provide for fluid communication between the expansion control channel and the outlet.

22. The plug of claim 18, wherein the plug further comprises a frit positioned in the flow path prior to the outlet.

23. The plug of claim 18, wherein the plug further defines an exit channel positioned in the flow path between the expansion control channel and the outlet.

24. The plug of claim 23, wherein the exit channel is defined by a groove in an outer wall of the outer plug member and an inner wall of a reservoir following insertion of the plug into a reservoir of a drug delivery device, and wherein a passage in a wall of the outer plug member provides for fluid communication between the expansion control channel and the exit channel.

25. The plug of claim 23, wherein the exit channel comprises a flowable material immiscible with the formulation to be delivered through the flow path.

26. The plug of claim 23, wherein the plug further comprises a frit positioned in the flow path between the exit channel and the outlet.

27. The plug of claim 18, wherein the expansion control channel comprises a first channel extending longitudinally through the inner plug member, a second channel extending laterally through the inner plug member, and a third helical channel extending in a spiral fashion along or through the inner plug member.

28. The plug of claim 27, wherein the third helical channel is defined by mating surfaces of an outer wall of the inner plug member and an inner wall of the outer plug member.

29. A drug delivery device comprising:
a reservoir body defining a reservoir for retaining a formulation comprising a drug; and
a plug according to claim 1;
wherein the plug is seated within the reservoir to provide for a flow pathway from the reservoir and out the outlet of the plug.

30. The drug delivery device of claim 29, wherein the drug delivery device is implantable.

31. The drug delivery device of claim 29, wherein the drug is selected from the group consisting of peptides, polypeptides, nucleic acids, and hormones.

32. The drug delivery device of claim 29, wherein the drug delivery device is operably attached to a catheter for delivery of the formulation from the reservoir to a delivery site.

33. A method of delivering a drug to a delivery site, the method comprising:
implanting at least a portion of drug delivery device in a subject, the drug delivery device comprising a plug according to claim 1 and a reservoir body defining a reservoir for retaining a formulation comprising a drug, wherein the plug is seated within the reservoir to provide for a flow pathway from the reservoir and out the outlet of the plug;
delivering the formulation from the reservoir and to a delivery site in a subject.

34. The method of claim 33, wherein the drug delivery device further comprises a catheter operably attached for delivery of the formulation from the reservoir and to the delivery site, wherein at least a distal end of the catheter comprising a drug delivery outlet is implanted in the subject.

35. The method of claim 33, wherein the drug is selected from the group consisting of peptides, polypeptides, nucleic acids, and hormones.
